(19) United States
(12) Patent Application Publication
(10) Pub. No.: US 2007/0260220 A1
(43) Pub. Date: Nov. 8, 2007

(54) Quick-attachment device for catheters

(75) Inventors: Oliver GOEDJE, Grunwald (DE); Thomas THALMEIER, Dorfen (DE); Holger DIETHELM, Munich (DE)

Correspondence Address: WILLIAM COLLARD;COLLARD & ROE, P.C., 1077 NORTHERN BOULEVARD, ROSLYN, NY, 11576, US

(73) Assignee: Pulsion Medical Systems AG

(21) Appl. No.: 11/799,478

(22) Filed: May 1, 2007

(30) Foreign Application Priority Data
May 2, 2006 (EP) 06113359.1

(51) Int. Cl. A61M 25/00

(52) U.S. Cl. 604/523

(57) ABSTRACT

A holder of the quick-attachment device integrated into the channel separation of a catheter to be fixed in place is deflected out and placed on the patient. By moving the holders into a position in which they are deflected out to a lesser degree, a shape-fit and/or force-fit connection is created between the patient and the quick-attachment device, for example by means of hooking into the skin. A counter-force prevents the holder from unintentionally being deflected again, to such a degree that the quick-attachment device could come loose from the patient, after having been fixed in place. The channel separation and the quick-attachment device form a unit that is structured or assembled in such a manner that channel separation and quick-attachment device do not fall apart during handling of the catheter in accordance with its intended purpose, i.e. do not unintentionally separate from one another.

### BACKGROUND OF THE INVENTION

1. Field of the Invention

The present invention relates to a quick-attachment device for catheters, particularly intravascular catheters.

2. The Prior Art

Intravascular catheters are used in medicine, particularly intensive care medicine, in many different ways. As a blood vessel access, they supply substances to the blood stream, for introducing measurement probes, for taking samples, as well as for setting up measurement points for pressure and temperature. Frequently, a catheter possesses more than one lumen, in order to be able to perform different functions (for example measuring blood pressure and taking blood samples). Distal to a branching referred to as a channel separation, these lumens run in a common catheter body; proximal to the channel separation, the lumens run in separate continuations.

To lay an intravascular catheter, the blood vessel is punctured, and subsequently the catheter is introduced, depending on the application after introduction of a guide wire and dilatation (widening) of the puncture point, for example, according to the so-called Seldinger technique. Since the catheter generally remains in the blood vessel over an extended period of time, in most cases for days, or at least for several hours, it is necessary to fix it in place on the patient, in order to prevent the position of the catheter in the blood vessel from changing, for example when the patient moves: Movement of the catheter farther out of the blood vessel would have the result that openings, sensor measurement points, etc. situated at the distal catheter end no longer exercise their function at the location originally provided; movement of the catheter farther into the blood vessel additionally bears with it the risk of infection. Furthermore, excessive movement of the catheter at its location of passage through the patient's tissue would further injure the tissue. Above all, however, the catheter should not be pulled entirely out of the blood vessel. Specifically in the case of an arterial catheter, this would mean significant blood loss at the puncture site.

Usually, catheters are sutured on, in order to fix them in place on the patient. Frequently, eyes through which the suture is drawn are provided for this purpose, in the region of the channel separation of the catheter, which is understood to be, as stated above, the switch-like transition between the distal catheter body and two or more proximal catheter connections.

Time must be expended for suturing, and this can delay other medical care. This is particularly problematic in the case of emergency patients. Two hands are needed for suturing, and under some circumstances, this can have the result that an additional helper is needed in order to perform other important manipulations at the same time. Furthermore, sterile suture material must be kept on hand.

Until now, the expenditure of time and suture material for placing an intravascular catheter was considered to be unavoidable.

### SUMMARY OF THE INVENTION

In view of the set of problems described above, it is an object of the invention to allow acceleration of catheter fixation.

According to one aspect of the invention, this task is accomplished by a quick-attachment device for catheters comprising holders that can be deflected, for holding the catheter on a patient, and means that exert a counter-force counter to further outward deflection of the holders, at least in one position of the holders. The quick-attachment device is integrated into a catheter channel separation.

The holders can have hooks for hooking into the skin of the patient. The hooks are preferably structured from stainless steel. The hooks can be made of plastic, mantled with plastic, or coated with plastic. The hooks are preferably sterile.

The means that exert a counter-force counter to further outward deflection of the holders, at least in one position of the holders, may comprise engagement means.

In another embodiment, the means that exert a counter-force counter to further outward deflection of the holders, at least in one position of the holders, comprise spring means that exert a re-set force on the holders when the holders are in the deflected-out state.

The device can also comprise clamping means that comprise the holders and spring means. The holders can be deflected out by means of spreading the clamping means. In another embodiment, the holders can be deflected out with one hand. There can be two pressure surfaces that can be pressed toward one another, for deflecting out the holders.

The invention also comprises a catheter that is equipped with a quick-attachment device according to the invention.

The invention also comprises an arrangement that comprises a catheter having a channel separation and a quick-attachment device, having holders that can be deflected out, for holding the catheter on a patient, and means that exert a counter-force counter to further outward deflection of the holders, at least in one position of the holders. The quick-attachment device has connecting means for holding the quick-attachment device on the catheter channel separation, and the channel separation is adapted for connecting with the connection means of the quick-attachment device.

The channel separation preferably has a groove for affixing the quick-attachment device to the catheter channel separation, and the quick-attachment device is adapted for holding in the groove of the catheter channel separation.

The holders of the quick-attachment device integrated into the channel separation of a catheter to be fixed in place are deflected and placed onto the patient. By moving the holders back into a position in which they are deflected to a lesser degree, a shape-fit and/or force-fit connection between the patient and the quick-attachment device is created, for example by means of hooking into the skin. A counter-force applied by suitable means prevents the holders from unintentionally being deflected, after fixation, to such a degree that the quick-attachment device could come loose from the patient. Advantageously, this can be achieved by means of engagement or by the re-set force of springs that are used.

Without the use of suture material, secure fixation of the catheter on the patient can thereby be achieved with a few hand movements. Particularly in the case of a clamp-like or vise-like structure of the holders, it is possible to use a one-hand operation, which is preferably made possible. Particularly when caring for emergency patients, one thereby achieves a valuable savings of time, as compared with the state of the art, and less distraction of the physician or medical personnel providing care.

As explained above, the quick-attachment device is integrated into the channel separation of a catheter, so that a common part, quick and easy to handle, is present. Integrated, in this connection, is understood as both fully integrated and partially integrated. In this connection, integrated means, in particular, that the channel separation and the quick-attachment device form a unit that is structured and/or joined together in such a manner that channel separation and quick-attachment device do not fall apart when the catheter is handled as intended, i.e. do not unintentionally separate from one another.

It is also in accordance with the invention to equip a quick-attachment device with connectors for holding the quick-attachment device on a catheter channel separation, and to provide a catheter channel separation specifically adapted for connecting with the connectors of the quick-attachment device.

Fundamentally, any variant of the invention described or indicated within the scope of the present application can be particularly advantageous, depending on the economic and technical conditions in an individual case. Unless something to the contrary is stated, and to the extent that this can fundamentally be implemented technically, individual characteristics of the embodiments described can be interchanged or combined with one another.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a top schematic view of the region of the channel separation of a catheter having a quick-attachment device according to one embodiment of the invention, configured in clamp-like manner;

FIG. 2 shows, schematically, the region of the channel separation of a catheter having a quick-attachment device similar to that in FIG. 1, configured as a double clamp;

FIGS. 3a-d illustrate the use of the quick-attachment device shown in FIG. 1;

FIG. 4 shows a perspective view of a variant of the connection of a quick-attachment device configured similar to FIG. 1 with the channel separation of a catheter;

FIG. 5 shows a perspective view of a variant of the integration of a quick-attachment device configured similar to FIG. 1 into the channel separation of a catheter;

FIG. 6 shows an alternative embodiment of a quick-attachment device according to the invention, which can be connected with conventional channel separations having suture eyes; and

FIG. 7 shows, schematically, the region of the channel separation of a catheter according to the state of the art.

### DETAILED DESCRIPTION OF THE PREFERRED EMBODIMENT

As shown in FIG. 7, which shows the state of the art, eyes 2 are often provided at the channel separation 1 of an intravascular catheter, for pulling through the suture with which the catheter is sutured to the patient. Distal catheter body 3 as well as proximal catheter connections 4 are shown only in part.

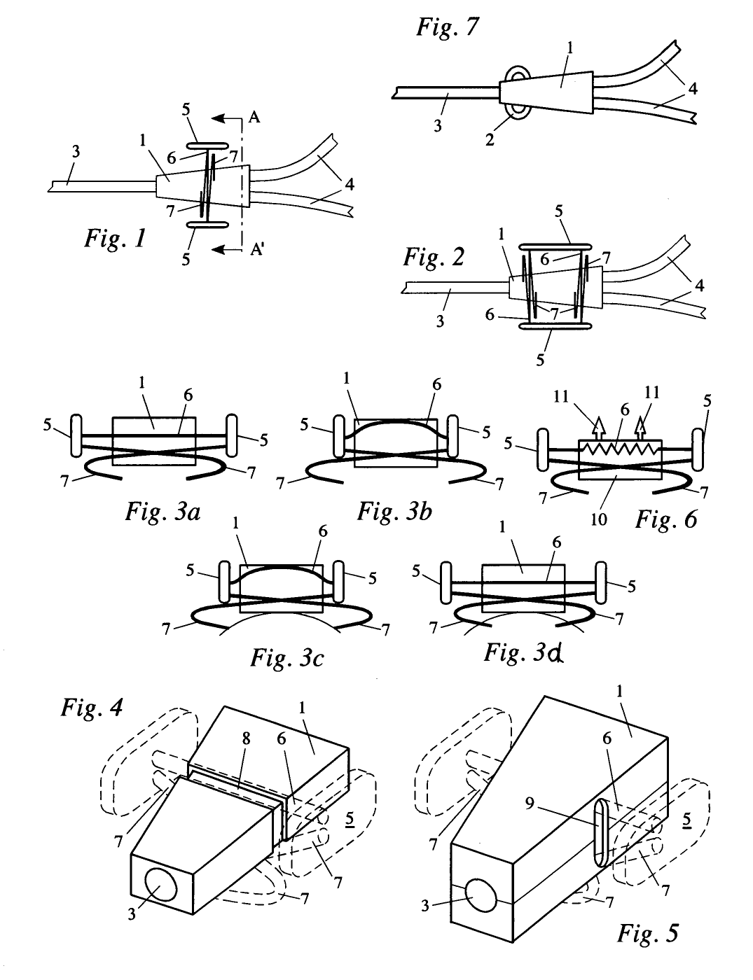

Channel separation 1 in FIGS. 1 and 2 (indicated with thinner lines, to show a better overview), having distal catheter body 3 and proximal catheter connectors 4, essentially corresponds to the state of the art, but is equipped with a quick-attachment device according to the invention. Eyes are not provided in the embodiment shown; according to one of the variants shown in FIGS. 4 and 5, the quick-attachment device can be connected with the channel separation 1. However, quick-attachment devices according to the invention can also be designed in such a manner, as described below using FIG. 6, that they are connected, in stable manner, with conventional channel separations having suture eyes.

The quick-attachment device shown in FIG. 1 has two plates with pressure surfaces 5 that lie on the outside, the spring 6 structured in the form of a bending rod, as well as two sterile stainless steel hooks 7. In place of stainless steel hooks 7, hooks of a different material or material combination can also be provided. Spring 6 and stainless steel hooks 7 together form a clamp. In this connection, spring 6 can be a tongue of spring steel that is separately inserted into the two plates, for example. However, spring 6 and stainless steel hooks 7 can also be bent out of a single piece of material, together.

In the case of the quick-attachment device shown in FIG. 2, two clamps as structured in FIG. 1 are provided, which are operated together (with one hand), by way of the pressure surfaces 5.

FIGS. 3a-d illustrate the method of functioning of the quick-attachment device from FIG. 1. The plane of representation is rotated by 90 degrees, as indicated in FIG. 1 by the dot-dash line A-A′, whereby the arrows indicate the viewing direction. The quick-attachment device from FIG. 2 functions in the same manner.

When pressure surfaces 5 are pressed towards one another, the spring 6 bends, and the sterile stainless steel hooks 7 are deflected out of their original position, as is evident in FIG. 3b. With the stainless steel hooks 7 deflected out, the quick-attachment device is placed against the skin of the patient (indicated with a thin, curved line), as shown in FIG. 3c. After the pressure surfaces 5 are let go, the spring 6 attempts to go back into its starting position. Via the re-set force of spring 6, stainless steel hooks 7 hook into the skin of the patient, as indicated in FIG. 3d, with their ends, which are sharpened for this purpose.

FIGS. 4 and 5 show two examples of a connection between the clamp of a quick-attachment device structured as in FIG. 1 and channel separation 1 of the catheter to be fixed in place. The projection of distal catheter body 3 is only indicated. As is usual according to the state of the art, it is practical to structure the housing of the catheter separation as an injection-molded part. Of course, other production methods are also possible.

For purposes comprehensibility, the quick-attachment device is shown with broken lines in FIGS. 4 and 5.

In FIG. 4, channel separation 1 possesses a circumferential groove 8. Even though this is shown in U shape, it can just as well be structured with a wedge shape or with a rounded cross-section. The quick-attachment clamp is pushed over channel separation 1 from the front during assembly. The re-set force of spring 6 then holds it in groove 8, in which hooks 7 also partly come to rest, so that channel separation 1 and the quick-attachment clamp are securely connected with one another, so that channel separation 1 and quick-attachment clamp remain against one another in medical use, during handling of the catheter, and do not fall apart.

With the variant shown in FIG. 5, which is not structured quite as flat, spring 6 and hooks 7 are guided by the injection-molded housing of channel separation 1. For this purpose, the injection-molded housing has lateral slits 9, and the quick-attachment clamp is already set into the housing during its production.

A hybrid version of the variants shown in FIG. 4 and FIG. 5, in which stainless steel hooks 7 run through the injection-molded housing of channel separation 1, and spring 6 comes to lie in a groove 8 or also simply on the flat top of the channel separation, is also possible. Such a solution allows a relatively flat structure of the channel separation 1.

FIG. 6 shows another embodiment of a quick-attachment device according to the invention, in a view analogous to FIG. 3a, but the channel separation has not been shown in this figure. Instead of a bending rod, spring 6 is structured as a helical spring. The quick-attachment device possesses its own flat housing 10, which is not identical with the housing of the channel separation of the catheter to be fixed in place. Two mandrels 11, which preferably consist of a rubber-elastic material, are disposed on housing 10. Mandrels 11 can be anchored in bores in housing 10, for example (not shown). A conventional channel separation 1, structured as in FIG. 7, is connected with the quick-attachment device by mandrels 11, in that mandrels 11 are inserted through its eyes 2. In place of mandrels 11, simple wire pins, for example, could also be provided, which are bent after having been passed through eyes 2. Here again, there is a secure connection, so that channel separation 1 and quick-attachment clamp remain on one another during handling of the catheter, in medical use, and do not fall apart.

Accordingly, while only a few embodiments of the present invention have been shown and described, it is obvious that many changes and modifications may be made thereunto without departing from the spirit and scope of the invention.

What is claimed is:

1. A quick-attachment device for catheters, comprising:
at least one holder that can be deflected, for holding the catheter on a patient; and
means that exert a counter-force counter to further outward deflection of the holder, at least in one position of the holder,
wherein the quick-attachment device is integrated into a catheter channel separation.

2. A quick-attachment device according to claim 1, wherein said at least one holder comprises hooks for hooking into the skin of the patient.

3. A quick-attachment device according to claim 2, wherein the hooks are structured from stainless steel.

4. A quick-attachment device according to claim 2, wherein the hooks are made from plastic, are mantled with plastic, or are coated with plastic.

5. A quick-attachment device according to claim 2, wherein the hooks are sterile.

6. A quick-attachment device according to claim 1, wherein the means that exert a counter-force counter to further deflection of the holder, comprise engagement means.

7. A quick-attachment device according to claim 1, wherein the means that exert a counter-force counter to further deflection of the holders, comprises a spring that exerts a re-set force on the holder when the holder is in a deflected state.

8. A quick-attachment device according to claim 7, further comprising a clamp that includes the holder and spring.

9. A quick-attachment device according to claim 8, wherein the holder can be deflected by spreading the clamp.

10. A quick-attachment device according to claim 1, wherein the holder can be deflected with one hand.

11. A quick-attachment device according to claim 10, further comprising two pressure surfaces that can be pressed toward one another, for deflecting the holder.

12. A catheter that is equipped with a quick-attachment device according to claim 1.

13. An arrangement comprising:
a catheter having a channel separation; and
a quick-attachment device, comprising:
at least one holder that can be deflected, for holding the catheter on a patient; and
means that exert a counter-force counter to further deflection of the holder, at least in one position of the holder;
wherein the quick-attachment device has connectors for holding the quick-attachment device on the catheter channel separation, and the channel separation is adapted for connecting with the connector of the quick-attachment device.

14. An arrangement according to claim 13, wherein the channel separation has a groove for affixing the quick-attachment device to the catheter channel separation, and the quick-attachment device is adapted for holding in the groove of the catheter channel separation.
